(19) United States
(12) Patent Application Publication
(10) Pub. No.: US 2026/0345096 A1
(43) Pub. Date: Nov. 5, 2026

(54) SMART WEARABLE HEALTH AND LOCATION MONITORING FOR CORRECTIONAL FACILITY INMATES

(71) Applicant: HLFIP Holding, LLC, Atlanta, GA (US)

(72) Inventor: Jonathan D. Logan, St. Petersburg, FL (US)

(73) Assignee: HLFIP Holding, LLC, Atlanta, GA (US)

(21) Appl. No.: 19/744,511

(22) Filed: Jul. 16, 2026

Related U.S. Application Data
(63) Continuation-in-part of application No. 19/217,435, filed on May 23, 2025.
Application No. 19/217,435 is a continuation of application No. 17/580,462, filed on Jan. 20, 2022, now Pat. No. 12,310,762.
(60) Provisional application No. 63/139,778, filed on Jan. 20, 2021.

(51) Int. Cl. A61B 5/00

(52) U.S. Cl. CPC A61B 5/746; A61B 5/0022; A61B 5/681; A61B 5/7405; A61B 5/742; A61B 5/7465

(57) ABSTRACT

The invention is a smart wearable device for inmates in a correctional facility. The device, non-removable by the wearer, utilizes a long-life battery, eliminating the need for cables or wires. The device is equipped with hardware and software for data gathering, including physiological and location tracking data, aiding in inmate management and custodial duties. The device enables real-time monitoring of inmate activities, health status, and communications, promoting quicker response times in health emergencies and assisting in the prevention and investigation of inappropriate or criminal behavior. The device also supports monitoring of macro-scale health patterns across different inmate populations, aiding in the identification of large-scale health concerns.

### CROSS-REFERENCE TO RELATED APPLICATIONS

This application is a continuation-in-part of U.S. Patent Application 19/217,435, filed May 23, 2025, titled “SMART WEARABLE DEVICE FOR TRACKING AND MONITORING INDIVIDUALS IN A CORRECTIONAL FACILITY” which is a continuation of U.S. Patent Application 17/580,462, filed Jan. 20, 2022, titled “SMART WEARABLE DEVICE FOR TRACKING AND MONITORING INDIVIDUALS IN A CORRECTIONAL FACILITY.” which claims the benefit of U.S. Provisional Application 63/139,778, filed Jan. 20, 2021, titled “SMART WEARABLE DEVICE FOR TRACKING AND MONITORING INDIVIDUALS IN A CORRECTIONAL FACILITY.” The contents of the above identified applications are incorporated herein by reference in their entirety.

### BACKGROUND

### FIELD OF THE ART

The present disclosure is related to the field of technological wearables designed for tracking, monitoring, and managing inmates in a correctional facility.

### Discussion of the State of the Art

In correctional facilities, inmates may experience a wide spectrum of medical emergencies, including but not limited to drug overdoses, injuries from altercations, and more conventional health issues. When these instances arise, rapid acknowledgment and response are paramount to provide timely medical attention. However, current correctional facilities generally lack the ability to monitor the health of individuals in real time and to swiftly respond by sending personnel to inmates in need.

Another issue faced within these facilities is the necessity to identify the location of the individual in distress. In certain scenarios, it may be advantageous to know who was in the vicinity of the individual having the medical emergency, as there are possibilities that other inmates may have contributed to the issue, either by providing illicit substances or inflicting harm. Existing facilities generally lack the monitoring capabilities to track the whereabouts of different individuals in relation to a medical emergency, making it arduous to determine who may have been involved in an incident.

Additionally, some medical emergencies occur outside of typical active monitoring hours such as when inmates are alone in their cells. In these scenarios, the existing monitoring approaches may not detect the medical emergency in time to react and provide necessary treatment.

Prior attempts to solve these problems have involved in-person monitoring, such as in-house guards, and video surveillance. However, these measures have proven insufficient as inmates can find ways to circumnavigate these systems, such as avoiding cameras or guards, or choosing times and locations where monitoring may be reduced or absent. This results in a suboptimal solution to the problems at hand.

### SUMMARY

The invention comprises a smart wearable device designed for use in correctional facilities. This wearable is configured to be non-removable by the inmate, incorporating a locking mechanism which cannot be unlocked by inmates and may be unlockable by facility or authorized personnel. Utilizing a long-life battery pack, which may also be non-removable by the wearer under general daily use conditions, the device maintains sufficient charge without the need for cables or wires. The battery pack may be removable by an inmate under certain circumstances such as when in proximity to a charging station or battery pack exchange kiosk or station.

The device is further equipped with hardware and/or software to collect and transmit data, including but not limited to physiological and location tracking information. This data assists in the effective management of inmates and helps perform custodial duties more efficiently. The smart wearable offers the capability to monitor inmate activities, health status, and communications, which facilitates prompt response to health emergencies and aids in the prevention and investigation of inappropriate or criminal behavior within the correctional facility.

The present invention surmounts the limitations of previous solutions by providing real-time health and location monitoring of each inmate. When a medical emergency is detected, the smart wearable can alert emergency personnel, providing them with the exact location of the inmate in distress to ensure immediate medical attention.

The benefits of the invention are manifold. It enhances inmate monitoring capabilities, facilitates rapid response to health emergencies, and aids in the prevention and investigation of inappropriate or criminal behavior. Compared to previous methods, this invention offers significant improvements in the management and monitoring of inmates within correctional facilities.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a network architecture for an exemplary smart wearable tracker in accordance with an exemplary embodiment of the invention.

FIG. 2 illustrates a system architecture for smart wearable tracker in accordance with an exemplary embodiment of the present invention.

FIG. 3 illustrates an exemplary computer implemented process for smart wearable tracker according to one embodiment of the invention.

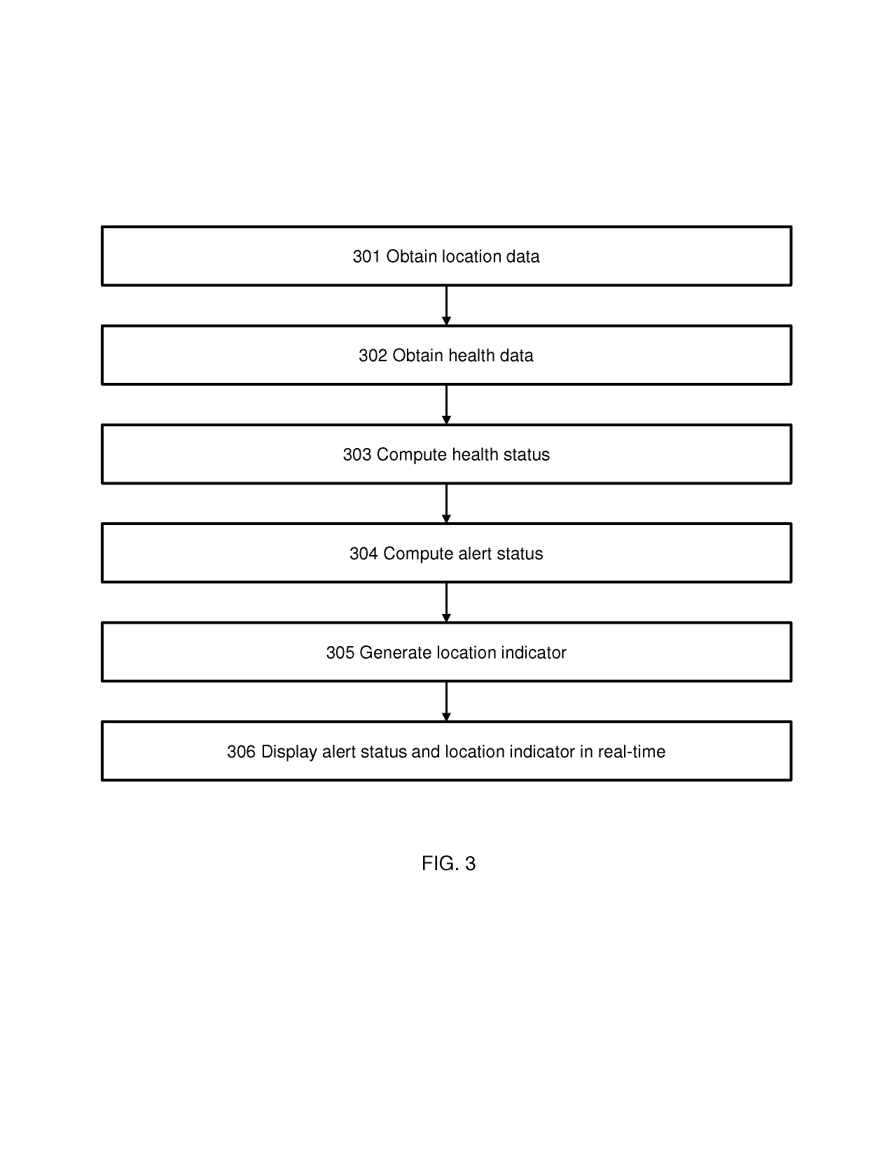

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Network Architecture

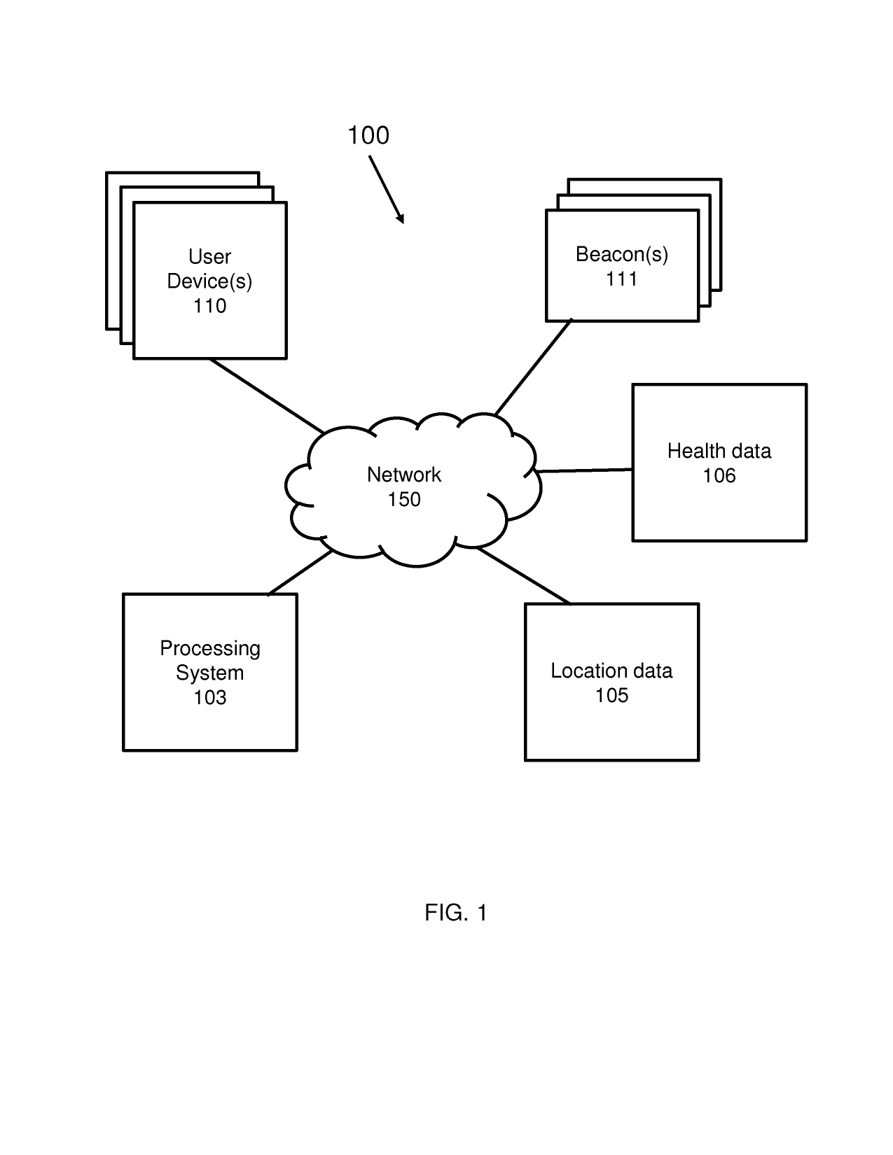

FIG. 1 illustrates an exemplary health monitoring and location tracking system according to an embodiment of the invention. The system comprises user device(s) 110, beacon(s) 111, location data 105, health data 106, processing system 103, and network 150. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

At a high level, the user device 110 is a smart wearable device designed to be affixed to a person’s body. This device can take various forms, such as a watch, wrist band, bracelet, ankle bracelet, arm band, finger-worn ring, or even a larger device that spans a significant portion of a wearer’s limb. The wearable device 101 is equipped with a range of components that gather data about the wearer and their surrounding environment.

The device functions by utilizing a variety of components like a location tracking unit, a microphone, a video camera, and a physiological data acquisition unit and/or sensors. The location tracking unit tracks the wearable’s location within and outside a facility, often through communication with beacons spread throughout the facility. The microphone captures audio information from the wearer and their surroundings, while the video camera captures visual information. The physiological data acquisition unit and/or sensors collect physiological data from the wearer. All of this data can be transmitted via network 150 for analysis by the processing system 103 and stored in a datastore for future access and analysis.

The wearable device 101 is designed to be tamper-proof to prevent inmates from disabling its functionality or using its internal components for undesirable purposes, such as using the battery or electrical components to start a fire. The wearable includes a securing mechanism that prevents an inmate from removing the wearable themselves. Control over this securing mechanism is given to facility personnel, not inmates.

In some aspects, user device(s) 110 or wearables could have different styles or capabilities based on the characteristics of the intended user or wearer. For instance, a certain monitoring device and/or securing mechanism might be utilized for high-risk or unstable inmates, while a different device and/or securing mechanism may be used for low-risk or minimal security inmates. This approach allows for customization and flexibility to fit the needs of various inmate populations.

At a high level, the system incorporates beacons 111 that serve as signal communication devices. These beacons interact with the user device(s) or wearable(s) to gather signal information for establishing the location of the user device relative to a particular beacon or plurality of beacons. Upon activation or when a location-determining request is made, one or more beacons initiate a signal communication protocol with one or more user devices. Each beacon then processes the raw signal data to ascertain its proximity to the wearable.

Alternatively, the raw data can be transmitted to a processing system for further analysis to derive proximity information. Data from multiple beacons, such as three beacons, can be amalgamated to pinpoint the location of the wearable, which can be accomplished through triangulation or other signal processing techniques.

In one particular aspect, a beacon may periodically undertake a signal communication protocol to identify any wearable within its signalling range and supply the corresponding signal communication data for additional processing. This capability extends to on-demand communication with a given wearable.

The beacons are strategically placed at predetermined locations based on the floor plan of the facility. The locations of the beacons are determined by mapping a floor plan from an imported file associated with a floor plan. These beacons can accurately locate the user device to within several feet or even a few inches. This precise location allows for determining if other inmates or wearables were in proximity to an individual experiencing a medical emergency, thereby enabling further investigation into potential misconduct.

The beacons can include various signal communication devices such as ultra-wide band beacons, Bluetooth, wifi access points, and the like. These alternatives offer flexibility and adaptability in setting up the beacon system according to the unique requirements of each correctional facility.

At a high level, the system 100 is operable to use location data 105 to track and monitor the location of multiple user devices or wearables within a correctional facility. This system collates data associated with the location of numerous wearables and can, at any given time, derive location data for a specific wearable. The system hosts a database of historical location data, providing a reviewable log of inmate locations which can be valuable for investigative personnel and procedures following a medical emergency, for instance. Processing of location data 106 may be done by processing system 103 according to an embodiment of the invention.

Processing the location data generally works by collecting raw signal data from beacons within the facility, including time and date stamps associated with when the location signal data was acquired. This raw data can then be processed, either in real time or stored for later analysis, to derive location information. Alternatively, the raw signal data may have been previously converted into location data prior to being stored in the system.

The system is designed to be routinely updated with recent location information for multiple wearables, ensuring that the most accurate and up-to-date data is available at all times. This location data can be utilized to perform ongoing and real-time headcounts of inmates in the facility. In one or more embodiments, the system executes an automated headcount process. The automated headcount process programmatically generates a comprehensive headcount report at scheduled intervals, on-demand by facility personnel, and/or dynamically in response to specific security triggers. This report automatically aggregates, cross-references, and logs the precise date, time, and specific location data for every individual inmate within the facility. Simultaneously, the an automated headcount report may capture and embed the real-time health biometrics (including, but not limited to, heart rate, body temperature, or respiration rate) of each inmate. This ensures that not only is the physical presence and location of each inmate verified within the facility, but their live physiological baseline is actively documented to confirm their vital status during the specific headcount window.

Beyond simple headcounts, the system also enables biometric identity verification. This process involves gathering and evaluating health data for an individual, and comparing this health data with expected norms for that individual derived from previously acquired and monitored health data. This feature makes the system more reliable than traditional methods as the wearables are non-removable by inmates, and their presence on a user cannot be faked. The system can use the physiological data not only to verify that the wearable is affixed to a living individual, but also to aid in identifying the individual based on biometrics.

An alternative to this system could involve using different types of sensors or devices for tracking inmate locations. For instance, Radio Frequency Identification (RFID) tags or Global Positioning System (GPS) trackers could be used. However, these alternatives would still require a similar system to process and store the location data gathered.

At a high level, the system 100 processes health data 106 obtained via smart wearable device(s) by compiling, processing, and storing physiological data associated with the individual wearing the device. This data is collected from one or more sensors integrated into or associated with the wearable, and can either be raw signal data or processed data converted into standardized health metrics. The type of health metrics that can be tracked include, but are not limited to, heart rate, respiration rate, body temperature, stress level, sleep metrics, and exercise and activity metrics among others. In some scenarios, depending on the health conditions of a given user, additional metrics may be obtained and/or recorded which are useful in evaluating the particular health condition. For example, in the case of diabetes, the wearable may comprise a glucose sensor and blood sampling components to evaluate blood sugar status. This is merely one example, and the wearables and system may be adapted to address other health conditions as would be apparent to one of ordinary skill in the art.

The system works by actively updating a database with the recent health information for a plurality of wearables and their corresponding users. This allows for the generation of more comprehensive medical profiles for inmates. The health data 106 not only focuses on physical health but may also include mental health data. This is derived by analyzing inmate input and communications, such as journal entries, questionnaire responses, mood prompts presented to the user via wearable at different time points, and text and/or audio associated with communications transmitted via the wearable. The data may be processed using machine learning such as Large Language Models (LLM) and/or Latent Dirichlet Allocation (LDA) to determine user sentiment and derive a corresponding mental health metric and status.

An alternative to this system could involve the use of external sensors or devices that interact with the wearable to collect health data. Additionally, different forms of data processing algorithms could be used to convert raw signal data into health metrics. For the mental health aspect, an alternative could be using different Natural Language Processing (NLP) methodologies or sentiment analysis algorithms to determine user sentiment and derive mental health metrics.

At a high level, processing system 103 is a computing system designed to receive and process data from various sources to generate health alerts in case of detected medical emergencies. It includes the capability to process physiology or health data for a plurality of users, employing Artificial Intelligence (AI) models to determine baseline trends and patterns for each individual. The system can identify when physiology data deviates beyond expected ranges, either for individual users or the general population.

The operation of the processing system involves handling beacon signal information to calculate the location of the wearable device, potentially using triangulation or time-of-flight analysis of signals transmitted between beacons and wearables. The system is able to process location data derived from this beacon signal data.

Additionally, the processing system 103 also processes physiology data obtained from sensors associated with the user device. This data can be converted into various health metrics, such as heart rate, among others. The system then processes the health data derived from the physiology data, analyzing these computed health metrics to determine patterns, trends, and deviations, which may serve as an indication of a medical emergency.

Subsequently, the processing system 103 generates alerts when medical emergencies are detected. These alerts typically comprise an indication of the identified emergency as well as the location of the wearable associated with the emergency.

In terms of alternatives, the processing system 103 could potentially use other types of AI models to process the physiology or health data. Different methodologies for determining trends and patterns could be employed. Alternatively, other techniques for location tracking, such as Global Positioning System (GPS) or Wi-Fi signal strength, could be used in place of beacon signal triangulation or time-of-flight analysis. Different sensors could be used to collect the physiology data, and other health metrics could be computed and analyzed for potential indications of medical emergencies.

User device(s) 110 include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

### System Architecture

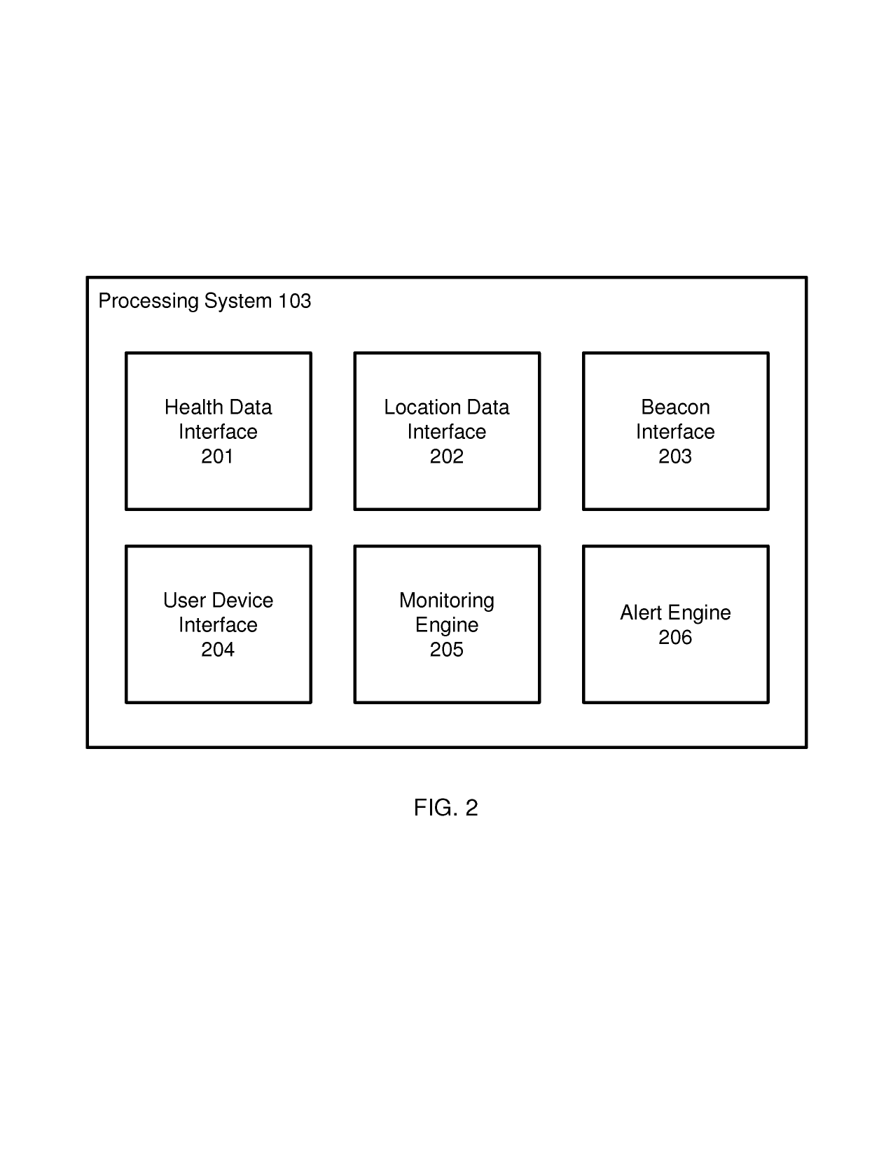

FIG. 2 illustrates an exemplary processing system for a health and location monitoring system for a smart wearable tracker according to an embodiment of the invention. The system comprises health data interface 201, location data interface 202, beacon interface 203, user device interface 204, artificial intelligence (AI) engine 205, and alert engine 206 The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

At a high level, health data interface 201 functions as an intermediary between the processing system and smart wearable devices worn by inmates and/or one or more external sources of health data. This subsystem is designed to access and process health metrics and physiology data associated with each inmate and/or their specific wearable device.

In operation, the health data interface 201 fetches these data elements, which may be stored in a designated database or obtained directly from a smart wearable(s). This process may involve sending requests to the database and/or wearable(s) to retrieve specific data or data entries, receiving the data, and then processing the data for further use within the system. In essence, the health data interface 201 serves as a conduit for health data, ensuring the necessary information is readily available for monitoring and responding to the health status of inmates.

In terms of its functionality, it can be programmed to extract data at regular intervals or in response to specific triggers or events. For example, it may request data when a new inmate is registered, when a wearable device signals an anomaly, or when a manual data pull is initiated by staff.

As for alternatives, while the health data interface 201 is described as interfacing with a database, it could also potentially interface with other types of data repositories or systems. For instance, it may connect directly to electronic health record systems or other healthcare information systems. The specific implementation can depend on the technology infrastructure of the correctional facility and the nature of the health data sources available.

At a high level, location data interface 202 is operable to interface with one or more external sources of location data associated with inmates and the corresponding wearables allocated to these inmates.

The function of location data interface 202 is to acquire current and/or historical location data, which are stored in a database. This operation allows for effective tracking of inmate movements both in the present and retrospectively, providing a comprehensive overview of the inmate’s location history.

The location data interface 202 operates by connecting with the external sources of location data, which could be the wearables themselves or other devices within the correctional facility that interact with these wearables, as well as a database of previously recorded location data. Once connected, it fetches the required data from these sources, either in real-time or from historical records, and stores this information in a dedicated database.

While the primary operation of the location data interface 202 involves interfacing with external sources of location data, it could also potentially interface with internal data sources within a correctional facility’s system. This could include other software subsystems or databases that store similar location data. This can serve as an alternative means of gathering location data if the primary external sources are unavailable or insufficient.

At a high level, the beacon interface 203 is responsible for communicating with one or more beacons to facilitate real-time location tracking. As part of its core functionality, it acquires and/or derives location data based on the information received from the beacons.

The beacon interface primarily acts as a communication interface, instructing beacons to ping the wearable device for location data and receiving corresponding signals associated with these requests. It does this by sending out a signal to beacons placed strategically within the correctional facility. The beacons then ping the wearable device and send back the signal information to the beacon interface.

In one aspect, data obtained from the beacon interface can be integrated with data from the user device interface to derive more precise and accurate location data and determine an individual’s location when a medical condition or emergency arises. This derived location data is then transmitted and stored in a location database, such as via location data interface 202.

Alternatively, while the primary function of the beacon interface is to communicate with beacons, similar functionality could potentially be achieved using other types of location tracking technologies such as GPS or RFID, depending on the specific requirements and constraints of the correctional facility.

At a high level, the user device interface 204 is operable to communicate directly with user devices, specifically wearables, to gather the data necessary for health monitoring and location tracking of the wearer. While the health and location data interfaces have been described above, the system may rely on the user device interface 204 to obtain the necessary health and location data directly from user devices, such as the smart wearables and/or other user devices associated with individual users.

In operation, the user device interface 204 is designed to initiate direct communication with the wearables either routinely or on demand. The process of communication involves obtaining specific data from the wearables, such as physiological metrics and/or geographical coordinates. This data aids in monitoring the health status and location of the wearer.

Upon acquisition of the data, the user device interface 204 processes the information and prepares it for transmission. The generated health and/or location data is then sent for storage via their corresponding interfaces, ensuring the data is safely stored and readily accessible for subsequent analysis or review.

In terms of alternatives, while the user device interface 204 is designed to communicate directly with wearables, it could also be adapted to communicate with other types of user devices. For instance, it may be configured to interface with handheld devices, biometric scanners, or other types of monitoring equipment. The key function remains the same - to gather necessary data for health monitoring and location tracking, albeit the source of the data may vary.

At a high level, monitoring engine 205, serves as the central processing unit for the acquired health and location data from the wearable device. It is designed to detect alterations in health status and trigger alerts when necessary.

The function of monitoring engine 205 is to analyze the collected health data, such as by applying various Artificial Intelligence (AI) learning and inferencing techniques. It uses AI algorithms to learn characteristics unique to a given individual, generating custom models with improved capabilities for determining changes in a specific user. By identifying patterns or trends indicative of a user moving towards a health complication and/or emergency, it serves as a proactive system for health monitoring. It applies thresholding to health data and derived health metrics to determine if health measures are abnormal or outside of an expected range, which could indicate a potential emergency.

In terms of operation, monitoring engine 205 processes the location data in combination with beacon and facility floor plan information to generate a mapped floor plan of the facility. This involves importing an image file associated with a floor plan for a building, defining a global origin based on the image file, generating polygons to define location areas associated with at least one tracking beacon, and categorizing location areas associated with each polygon. The beacons are positioned at predetermined locations based on the facility’s floor plan, with the locations determined by mapping a floor plan from an imported file associated with the floor plan.

An additional function of monitoring engine 205 is to perform remote wellness checks on individuals. These checks could be scheduled, periodic checks and/or on-demand wellness checks triggered by the system based on input received from a user. To support the execution of remote wellness checks, the user device 110 or smart wearable device incorporates one or more user-interactive inputs, such as a physical mechanical button, a tactile capacitive sensor, a touchscreen interface indicator, and/or a voice-activated audio prompt. The wearer can interact with this input to explicitly communicate their immediate status, such as selecting a first option or button sequence indicating that they are secure and healthy (e.g., an "I am OK" status signal) or a second option or button sequence indicating an immediate need for assistance (e.g., a "Help Needed" status signal). Furthermore, the monitoring engine 205 and user device interface 204 are configured to enable authorized facility personnel to initiate a remote, bidirectional video call directly to the smart wearable device. This enables a face-to-face video chat between the staff member and the wearer utilizing the device's integrated video camera and microphone. Upon initialization, the system automatically generates a secure cryptographic date and time stamp for the call, records the audio-visual streaming data of the session, and saves the compiled video recording directly into the inmate’s digital user profile. Concurrently, during the entirety of the active video call session, the system continuously streams and logs the inmate's real-time health biometrics, binding the physiological data to the recorded session within the inmate's profile to monitor physical distress or deceptive markers during the live interaction.

Alternative techniques, such as traditional data processing algorithms, could be used in place of AI for analyzing health and location data. Similarly, other mapping techniques could be used to generate the facility floor plan..

At a high level, alert engine 206 is designed to handle and manage alert generation in case of health complications or emergencies detected by the wearable device. It functions by producing a medical alert that comprises both an indication of the medical need and the corresponding location of the user in need.

The alert engine operates by generating an indicator for display on a graphical user interface. This indicator shows the location of the medical need in relation to a floor plan of the facility. The engine utilizes data input from the monitoring engine and/or the mapped floor plan to generate this location indicator. In one aspect, alert engine may generate audible alerts such as triggering an alarm sound to be generated by the wearable associated with a user requiring medical attention. Alert engine may generate audible alerts that can be played throughout a facility such as via broadcasting over a public address system. The alert may indicate the type of emergency and/or the location of the emergency.

In terms of functionality, when a health complication or emergency is detected, the alert engine springs into action. It creates a medical alert that not only signifies the occurrence of a medical emergency, but also pinpoints the exact location of the user in need. This dual-purpose functionality allows for a swift and accurate response to emergencies.

The alert engine can also adapt to different formats of location representation. For instance, instead of identifying a specific room or location on a floor plan, the alert could alternatively be configured to indicate a zone or sector within the facility. This flexibility allows the alert engine to cater to various facility layouts and emergency response protocols.

### Computer Implemented Process

The process steps described herein may be performed in association with a system such as that described in FIG. 1 and/or FIG. 2 above or in association with a different system. The process comprises obtaining location data 301, obtaining health data 302, computing health status 303, computing alert status 304, generating location indicator 305, displaying alert status and location indicator in real-time 306. The process may comprise additional steps, fewer steps, and/or a different order of steps without departing from the scope of the invention as would be apparent to one of ordinary skill in the art.

At 301, the software process involves obtaining location data associated with a user device, where the user device is a wearable item, such as a smart watch worn by the user. This location data is generated by beacons that are capable of identifying the location of the user device relative to a facility.

The location data may be derived from a mapped floor plan of at least a portion of the facility, along with data from at least one beacon. This mapping includes importing an image file associated with a floor plan for a building, defining a global origin based on the image file, generating a plurality of polygons to define location areas to be associated with at least one tracking beacon, and assigning location areas associated with each polygon to a category or classification. Positioning of the beacons at predetermined locations is determined based on the mapped floor plan of the facility. This determination involves mapping a floor plan from an imported file associated with a floor plan.

The beacons may comprise ultra wide band beacons, Bluetooth, and/or WiFi access points. These technologies offer respective advantages in terms of their range, accuracy, and power consumption, and can be selected based on the specific needs and constraints of the facility.

The location data includes data obtained at periodic intervals over time, which allows the generation of a set of historical locations of the user device. This historical data can be used to track the movement and activities of the user over time, supporting various monitoring and management functions within the facility.

Other types of beacons or positioning technologies could be used, such as RFID or GPS, depending on the indoor or outdoor nature of the facility and the level of location accuracy required. The floor plan could also be derived from other sources, such as CAD files or manual measurements, rather than image files. The wearable device could take other forms, such as a bracelet or a badge, rather than a smart watch.

At 302, the software process obtains health data associated with the user from the user device or wearable. This health data comprises physiological data of the user. The health data is acquired in real-time while the user is wearing the user device, ensuring up-to-date and continuous monitoring of the user’s physiological state. The health data may additionally comprise mental health data associated with the user. This data is useful in providing a holistic view of the user’s overall health, taking into account not only physical but also mental well-being.

The physiological data may comprise at least one of heart rate, body temperature, stress level, sleep, and activity. These data points provide a comprehensive overview of the user’s physical health and are valuable markers for various health conditions. The activity data may be generated from at least one of an accelerometer and an imported exercise equipment data set. This allows for accurate tracking of the user’s physical activity, contributing to the overall picture of the user’s health. The physiological data may be obtained through biometric sensors. These sensors provide precise and reliable measurements, enhancing the accuracy of the collected health data.

The mental health data of the user may be obtained through at least one of receiving user input through the user device and speech data. This could include phone call recording data, journaling data, etc., which may be processed via speech to text, and/or AI sentiment analysis. This step allows for an innovative approach to mental health monitoring, utilizing AI to analyze speech patterns and sentiments to infer mental well-being.

The health data may comprise at least one of a plurality of sets of health data associated with a user obtained at periodic intervals through the user device and/or data from an electronic medical records system (EMR). This integration allows for a comprehensive health profile of the user, combining real-time data from the wearable device with historical and clinical data from the user’s EMR.

Alternative methods of data collection and analysis could include using different types of sensors or data inputs, such as GPS for location tracking or additional biometric measures like blood oxygen levels or glucose levels. Mental health data could also be obtained through alternative methods such as self-report surveys or other behavioral data.

At 303, the software process involves computing a health status of the user based on acquired health data. This computation leverages predictive analytics applied to the health data for an accurate assessment of the user’s health status. The computation of health status includes trend analysis, which identifies typical patterns or characteristics unique to each individual user along with other biometric characteristics. This step aids in creating a personalized health profile for each user.At step number three, the software process continually monitors the health data of the user. The monitoring process involves processing the obtained health data to identify any adverse changes in the user’s health status and subsequently generate alerts if necessary.

The monitoring process may employ artificial intelligence (AI) learning and inferencing techniques to analyze health status. AI algorithms are adapted to learn characteristics unique to a given individual, leading to the generation of custom models with improved capabilities for determining changes in a specific user as compared with general population-based models.

The software process may look for and identify patterns or trends indicative of a user moving towards a health complication and/or emergency. This includes applying filtering and/or thresholding to health data and derived health metrics to determine if health measures are abnormal or outside of an expected range, which might indicate a potential emergency.

The software process may comprise performing remote wellness checks on individuals via analysis of health data. These wellness checks can be scheduled periodically or triggered on-demand by the system based on input received from a user. Certain individuals, such as high-risk inmates, may be subjected to more frequent wellness checks.

At 304, the software process involves computing an alert status which is achieved by evaluating the monitoring data to ascertain if an alert is warranted. This evaluation is based on the health status of the user and involves comparing the health status against a predetermined threshold or acceptable/normal ranges. If the health status crosses this threshold or falls outside the acceptable ranges, the system computes an alert status. This alert status is indicative of a medical condition associated with the user.

At step number two, the software process employs various data filtering techniques to compute the alert status. These techniques help to ensure fewer false alarms based on health monitoring while maintaining sufficient sensitivity to detecting true medical emergencies. This is achieved by applying user-specific thresholding to determine if an individual user's metrics are out of their normal range. This user-specific thresholding allows for personalized monitoring, taking into account the unique health parameters and condition of each user.

At step number three, the software process offers an alternative approach to computing an alert status. Instead of user-specific thresholding, general population norms or ranges can be used for comparison. This approach is particularly useful when individual user data is not available, or when the user's health status needs to be compared to a broader population for a more generalized health assessment.

At step number four, the alert status computed by the software process may include a severity indicator. This indicator is dependent on the urgency of the identified medical emergency, providing an immediate understanding of the criticalness of the situation and enabling appropriate and timely response.

At 305, the software process generates a location indicator based on the location data. This location indicator signifies the location of the user device in relation to the facility. It provides specific location information such as the zone, floor, room, hallway, or exterior location associated with the facility.

At step number two, the software process includes a method of generating the location indicator, which involves using a mapped floor plan of the facility. The software process processes location data in conjunction with beacon and/or facility floor plan information to generate a mapped floor plan of the facility.

At step number three, the mapped floor plan is generated by importing an image file linked with a floor plan for the building. The software process defines a global origin based on this image file.

At step number four, the software process generates a multitude of polygons to define location areas meant to be associated with at least one tracking beacon. This step involves assigning location areas associated with each polygon to a specific category or classification.

At step number five, the beacons are positioned strategically at predetermined locations according to the floor plan of the facility. The determination of the locations of the beacons involves mapping a floor plan from an imported file associated with a floor plan.

As an alternative to using an image file linked with a floor plan for the building, the software process could also employ a digital blueprint or a CAD model of the facility. Additionally, instead of using polygons to define location areas, other geometric shapes such as circles, rectangles, or even complex custom shapes could be used. The location areas could also be assigned to a hierarchy or a group, instead of a category or a classification. Lastly, the beacons could be positioned based on a 3D model of the facility, instead of a 2D floor plan.

At 306, the software process involves displaying, in near real-time, the alert status associated with a user and the location indicator associated with the user device on a graphical user interface. This serves to notify facility personnel about the location of the user device linked to a medical condition relative to the facility’s floor plan. In essence, the system is designed to provide immediate visual cues to the personnel about any health emergencies and the location of the affected individuals within the facility.

Proceeding to step number two, the software process expands to display, on the same graphical user interface, the historical location data of the user and/or other users in proximity to the user when the medical emergency occurred. This provides a retrospective tracking capability, enabling facility personnel to understand the movements of the affected user and those nearby leading up to the health emergency. This could potentially help identify any interactions or incidents that might have contributed to the medical condition.

Moving on to step number three, the software process also includes displaying location data for a plurality of different user devices. This feature furnishes real-time headcount and location data for individuals associated with the facility. Essentially, this facilitates efficient monitoring of the facility’s population, providing a live view of the whereabouts of all individuals within the facility.

Finally, at step number four, the software process provides location data for a plurality of different user devices associated with a location near the location indicator. This means that, in addition to the real-time location of the user experiencing a medical condition, the software can also provide location data for other users in close proximity to the affected individual. This can be particularly useful in identifying potential witnesses or contributors to the incident.

Alternative methods that can be used in the process could include displaying alerts in different formats or using different visual cues on the graphical user interface. Furthermore, the process could also incorporate different data collection or tracking methods to gather location and historical data, while maintaining the primary function of providing real-time location and alert status information.

Lastly, while described herein with particular applications to correctional facilities and inmates, the systems and processes disclosed can also be applied to monitor individuals in other settings such as mental health clinics, psychiatric hospitals, long term care facilities, and similar institutions. The system configuration and process remains generally the same albeit with adaptations for the specific context and/or environments where they are applied.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

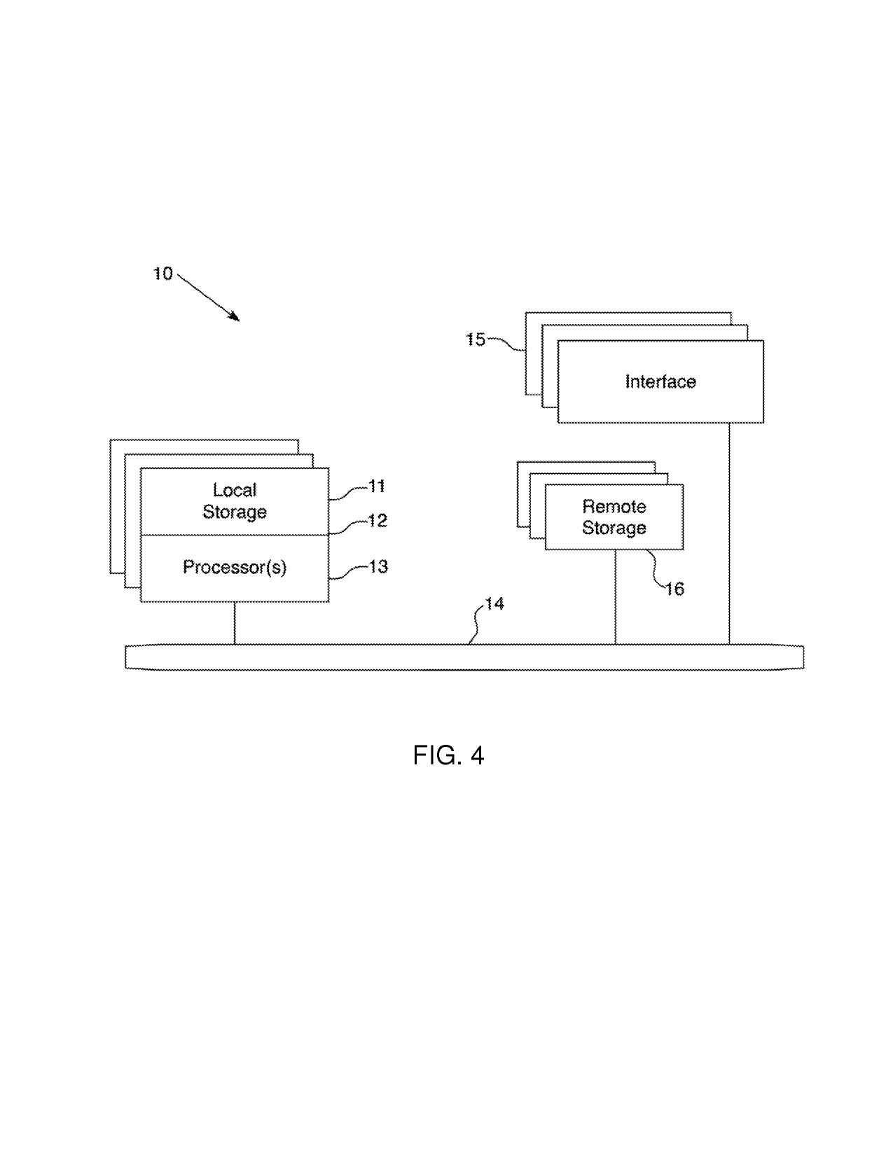

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components, process steps or the like may be and/or comprise hardware and/or software as described herein. For example, the systems, engines, and subcomponents described herein may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine- readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

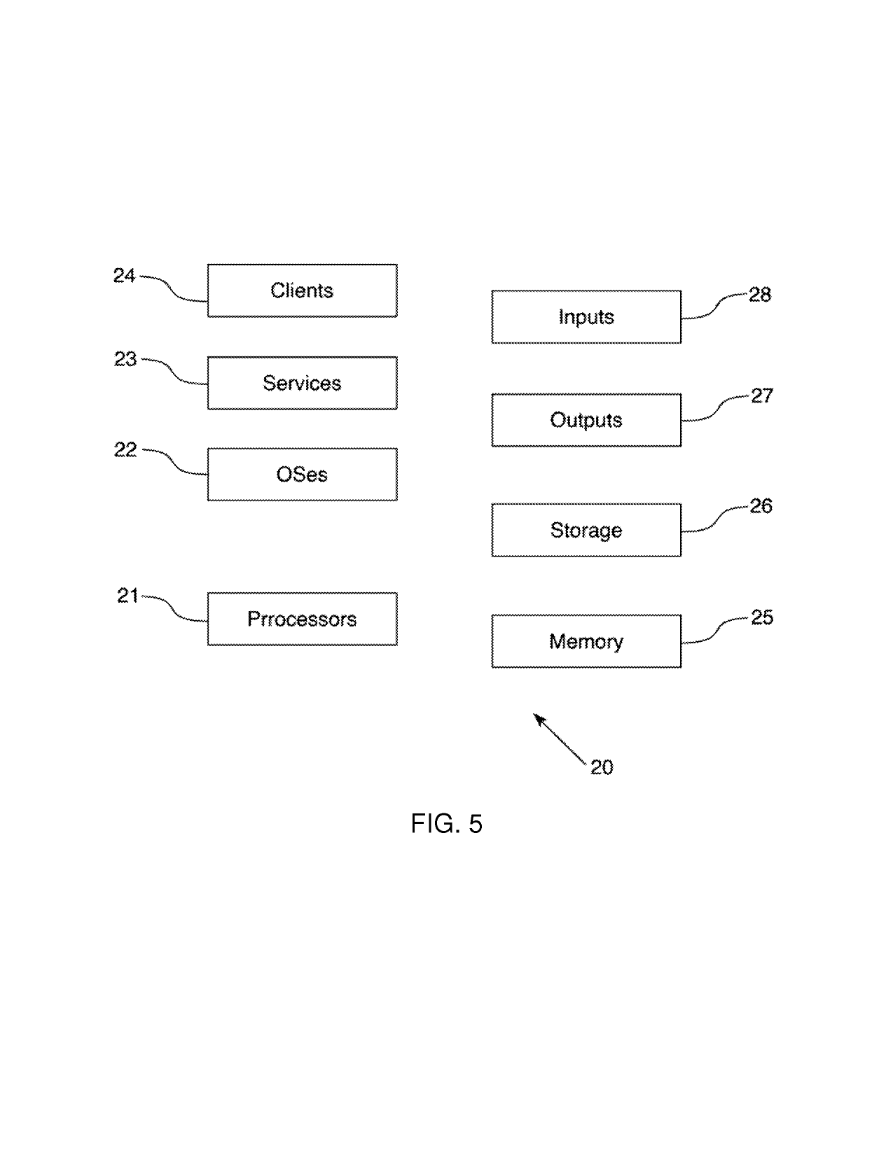

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

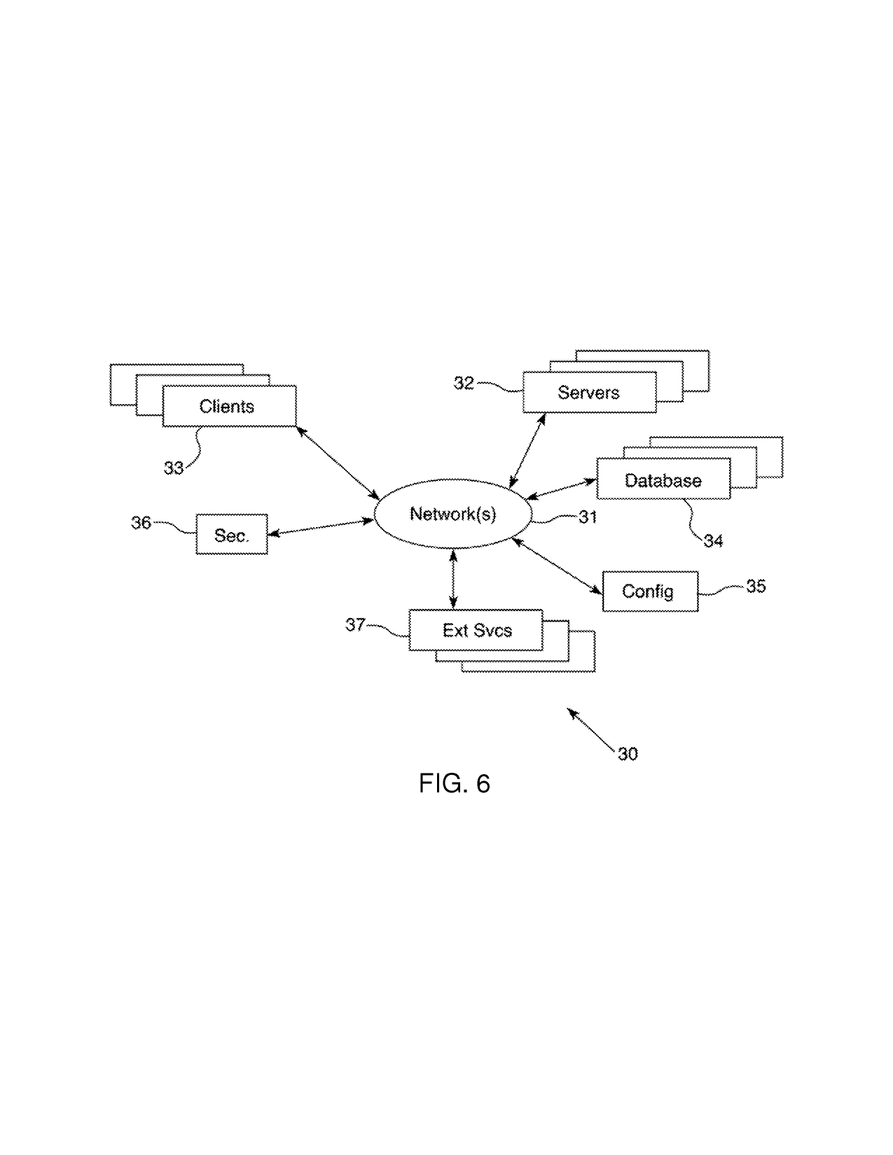

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise’s or user’s premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

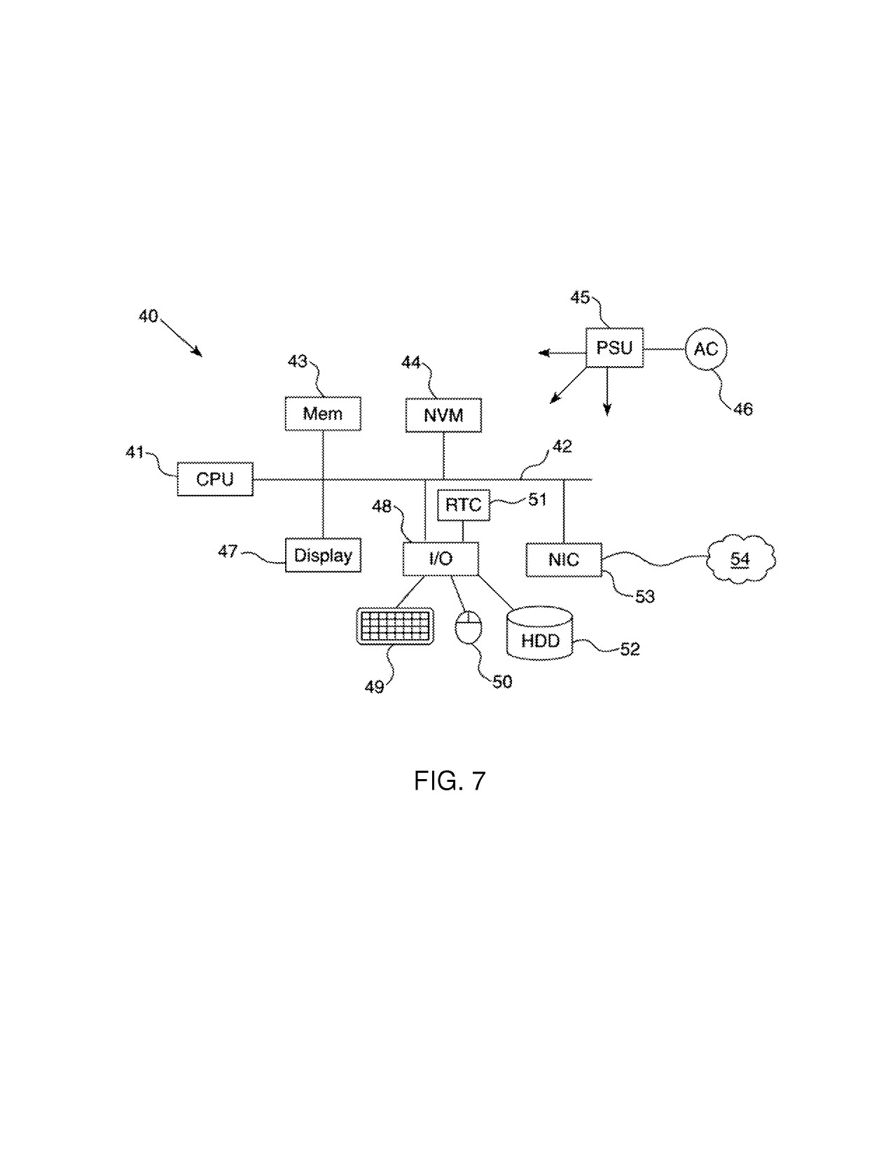

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to "one embodiment" or "an embodiment" means that a particular element, feature, structure, or characteristic described in connection with the embodiment is included in at least one embodiment. The appearances of the phrase "in one embodiment" in various places in the specification are not necessarily all referring to the same embodiment.

Some embodiments may be described using the expression "coupled" and "connected" along with their derivatives. For example, some embodiments may be described using the term "coupled" to indicate that two or more elements are in direct physical or electrical contact. The term "coupled," however, may also mean that two or more elements are not in direct contact with each other, but yet still co-operate or interact with each other. The embodiments are not limited in this context.

As used herein, the terms "comprises," "comprising," "includes," "including," "has," "having" or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, "or" refers to an inclusive or and not to an exclusive or. For example, a condition A or B is satisfied by any one of the following: A is true (or present) and Bis false (or not present), A is false ( or not present) and Bis true ( or present), and both A and B are true ( or present).

In addition, use of the "a" or "an" are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computer implemented method for real-time health monitoring and location tracking of facility residents, the computer implemented method comprising:
obtaining location data associated with a user device, wherein the user device comprises a device worn by the user, the location data generated by beacons capable of identifying the location of the user device relative to a facility, the location data derived from a mapped floor plan of at least a portion of the facility and data from at least one beacon;
obtaining health data associated with the user from the user device, wherein the health data comprises physiological data of the user, the health data acquired in real-time while the user is wearing the user device;
computing a health status of the user based on the health data, the health status of the user computed using predictive analytics applied to the health data;
computing an alert status by applying a threshold to the health status, the alert status indicating a medical condition associated with the user;
generating a location indicator based on the location data, the location indicator indicating the location of the user device relative to the facility, the location indicator indicating at least one of a zone, floor, room, hallway and exterior location associated with the facility and the location data; and
displaying, in near real-time, on a graphical user interface the alert status associated with the user and the location indicator associated with the user device, thereby alerting facility personnel to the location of the user device associated with the medical condition relative to the floor plan of the facility.

2. The computer implemented method according to claim 1, wherein the device worn by the user comprises a smart wearable or smart watch.

3. The computer implemented method according to claim 1, wherein the mapped floor plan is generated by importing an image file associated with a floor plan for a building; defining a global origin based on the image file; generating a plurality of polygons to define location areas to be associated with at least one tracking beacon; and assigning location areas associated with each polygon to a category or classification.

4. The computer implemented method according to claim 1, wherein the beacons are positioned at predetermined locations based on the floor plan of the facility, wherein determining the locations of the beacons comprises mapping a floor plan from an imported file associated with a floor plan.

5. The computer implemented method according to claim 1, wherein the beacons are capable of accurate placement of the user device to within inches.

6. The computer implemented method according to claim 5, wherein the beacons comprise ultra wide band beacons, BLUETOOTH, WiFi access points.

7. The computer implemented method according to claim 1, wherein the location data comprises data obtained at periodic intervals over time to generate a set of historical locations of the user device.

8. The computer implemented method according to claim 1, wherein the health data additionally comprises mental health data associated with the user.

9. The computer implemented method according to claim 1, wherein the physiological data comprises at least one of heart rate, body temperature, stress level, sleep, and activity.

10. The computer implemented method according to claim 9, wherein the activity data is generated from at least one of an accelerometer and an imported exercise equipment data set.

11. The computer implemented method according to claim 9, wherein the physiological data is obtained through biometric sensors.

12. The computer implemented method according to claim 8, wherein the mental health data of the user is obtained through at least one of receiving user input through the user device and phone call data associated with the user.

13. The computer implemented method according to claim 1, wherein the health data comprises at least one of a plurality of sets of health data associated with a user obtained at periodic intervals through the user device and data from an electronic medical records system (EMR).

14. The computer implemented method according to claim 1, additionally comprises displaying, on the graphical user interface, the historical location data of the user.

15. The computer implemented method according to claim 1, additionally comprises obtaining location data for a plurality of different user devices, thereby providing real-time headcount and location data for individuals associated with a facility.

16. The computer implemented method according to claim 15, additionally comprises providing the location data for a plurality of different user devices associated with a location near the location indicator.

17. The computer implemented method according to claim 1, further comprising establishing a bidirectional communication between the user device and facility personnel thereby enabling an on-demand, real-time wellness check with the user.

18. A computing system for real-time health monitoring and location tracking of facility residents, the computing system comprising:
at least one computing processor; and
memory comprising instructions that, when executed by the at least one computing processor, enable the computing system to:
obtain location data associated with a user device, wherein the user device comprises a device worn by the user, the location data generated by beacons capable of identifying the location of the user device relative to a facility, the location data derived from a mapped floor plan of at least a portion of the facility and data from at least one beacon;
obtain health data associated with the user from the user device, wherein the health data comprises physiological data of the user, the health data acquired in real-time while the user is wearing the user device;
compute a health status of the user based on the health data, the health status of the user computed using predictive analytics applied to the health data;
compute an alert status by applying a threshold to the health status, the alert status indicating a medical condition associated with the user;
generate a location indicator based on the location data, the location indicator indicating the location of the user device relative to the facility, the location indicator indicating at least one of a zone, floor, room, hallway and exterior location associated with the facility and the location data; and
display, in near real-time, on a graphical user interface the alert status associated with the user and the location indicator associated with the user device, thereby alerting facility personnel to the location of the user device associated with the medical condition relative to the floor plan of the facility.

19. A non-transitory computer readable medium comprising instructions that when executed by a processor enable the processor to:
obtain location data associated with a user device, wherein the user device comprises a device worn by the user, the location data generated by beacons capable of identifying the location of the user device relative to a facility, the location data derived from a mapped floor plan of at least a portion of the facility and data from at least one beacon;
obtain health data associated with the user from the user device, wherein the health data comprises physiological data of the user, the health data acquired in real-time while the user is wearing the user device;
compute a health status of the user based on the health data, the health status of the user computed using predictive analytics applied to the health data;
compute an alert status by applying a threshold to the health status, the alert status indicating a medical condition associated with the user;
generate a location indicator based on the location data, the location indicator indicating the location of the user device relative to the facility, the location indicator indicating at least one of a zone, floor, room, hallway and exterior location associated with the facility and the location data; and
display, in near real-time, on a graphical user interface the alert status associated with the user and the location indicator associated with the user device, thereby alerting facility personnel to the location of the user device associated with the medical condition relative to the floor plan of the facility.
